(19) United States
(12) Patent Application Publication
(10) Pub. No.: US 2018/0161304 A1
(43) Pub. Date: Jun. 14, 2018

(54) EQUINE SUPPLEMENT

(71) Applicants: Stanley N. Jankowitz, Franklin Lakes, NJ (US); Raj Chopra, Westbury, NY (US)

(72) Inventors: Stanley N. Jankowitz, Franklin Lakes, NJ (US); Raj Chopra, Westbury, NY (US)

(21) Appl. No.: 15/890,588

(22) Filed: Feb. 7, 2018

Related U.S. Application Data
(63) Continuation of application No. 14/215,870, filed on Mar. 17, 2014, now abandoned.
(60) Provisional application No. 61/802,624, filed on Mar. 16, 2013.

(51) Int. Cl. A61K 31/355; A61K 31/122; A61K 31/375; A23K 20/174; A23K 20/111; A23K 20/163; A23K 20/10; A23K 20/24; A23K 20/20; A23K 50/20; A61K 33/06; A61K 45/06; A61K 33/04

(52) U.S. Cl. CPC A61K 31/355; A61K 31/122; A61K 31/375; A23K 20/174; A23K 20/111; A23K 20/163; A61K 2300/00; A23K 20/24; A23K 20/30; A23K 50/20; A61K 33/06; A61K 45/06; A61K 33/04; A23K 20/10

(57) ABSTRACT

Disclosed herein are methods of using nutritional supplements and specific combinations thereof to counteract exercise-induced stress and its sequelae in members of the Equus species. Particular embodiments focus on use of Coenzyme Q10, vitamin E and their variants. Methods include ascertaining and modifying the nutritional supplements, ascertaining and comparing health states of a horse, wherein the comparisons are made before and after modifications of the supplements and/or implementation of exercise regimens.

### CROSS-REFERENCE TO RELATED APPLICATIONS

The present application is a continuation of U.S. application Ser. No. 14/215,870, filed Mar. 17, 2014, which claims priority to U.S. Provisional Patent Application Ser. No. 61/802,624, filed Mar. 16, 2013, the disclosures of each of which are incorporated herein by reference in their entirety.

### BACKGROUND

### Field of the Invention

Embodiments of the present invention are generally related to equine supplements and methods for supporting the health and wellbeing of horses and preventing and/or treating various physical and/or neurological conditions caused by deficiencies of vitamin E, or the like. More specifically, embodiments of the present invention relate to administering Coenzyme Q10, vitamin E, and other vitamins and minerals to thoroughbred race horses.

### Description of Related Art

Thoroughbred horses, race horses, and other types of horses often participate in high intensity stressful exercise, which may cause detrimental effects on the overall health and performance of the horses. The inflammatory response to vigorous exercise ranges from the symptoms of delayed-onset muscle soreness to more severe injuries affecting soft tissue, joint, and/or bone related to the depletion of Vitamin E. Vitamin E serves to maintain neurological health and normal neuromuscular function in horses. Deficiencies in vitamin E may lead to abnormal neuromuscular function and/or neurological diseases or disorders in horses. Some exemplary neurologic diseases that may affect horses due to a deficiency in vitamin E may include equine degenerative myeloencephalopathy (EDM) and equine motor neuron disease (EMND), to name a few. Many other neurological disorders may be linked to genetic or exercise-induced vitamin E deficiencies, and are contemplated by and within embodiments of the present invention. To prevent and/or treat these conditions, a need exists for improved equine synthetic and/or natural nutraceuticals and/or supplements and methods for supporting the health of horses and preventing physical and/or neurological disorders by improving the bioavailability and/or levels of Vitamin E in equines, or the like.

### SUMMARY

In one embodiment of the present invention a composition is provided that may comprise one or more of coenzyme Q10 and vitamin E. For example, a composition is provided that may comprise one or more of: an amount of coenzyme Q10 in the range of approximately 5 mg to approximately 1000 mg combined with an amount of vitamin E in the range of approximately 250 IUs to approximately 9000 IUs; an amount of Coenzyme Q10 of approximately 100 mg combined with an amount of vitamin E of approximately 1250 IUs; an amount of natural vitamin E in the range of approximately 1500 IUs to approximately 5000 IUs; an amount of Coenzyme Q10 in the range of approximately 250 mg and approximately 3.4 g; and an amount in a range between approximately 3.486 mg of Coenzyme Q10 per kg of body weight of a horse and approximately 6.238 mg of Coenzyme Q10 per kg of body weight of the horse.

Further, an additional embodiment of the present invention provides a method for ascertaining and comparing results of using nutraceuticals and/or nutritional supplements to counteract exercise-induced oxidative stress and its sequelae in a horse, the method comprising: determining dietary supplement and/or nutraceutical amounts for one or more of Coenzyme Q10 and vitamin E, wherein the dietary supplement amounts and/or nutraceutical amounts are based at least in part on one or more ascertained vital characteristics of the horse; administering to the horse a dietary supplement containing one or more of Coenzyme Q10 and vitamin E in the determined dietary supplement amounts; and ascertaining, with an equine blood-analysis apparatus, or the like, a health state of the horse, the ascertained health state of the horse being indicative of exercise-induced oxidative stress and its sequelae in the horse.

Another method in accordance with embodiments of the present invention comprising: modifying amount supplemented based at least in part on an ascertained health state of the horse; administering to the horse a nutraceutical and/or dietary supplement containing one or more of Coenzyme Q10 and vitamin E in the modified nutraceutical and/or dietary supplement amounts; ascertaining, with an equine blood-analysis apparatus, or the like, a health state of the horse, the ascertained health state of the horse being indicative of exercise-induced oxidative stress and its sequelae in the horse; and determining, with a computer, or the like, a result of a comparison between the ascertained health states, the determined result being indicative of using Coenzyme Q10 and vitamin E as dietary supplements to counteract exercise-induced oxidative stress and its sequelae in a horse.

Yet another embodiment of present invention may comprise implementing an exercise regimen for the horse, the exercise regimen comprising one or more sessions of physical activity, the physical activity comprising one or more of graduated training, low-intensity exercise or galloping, and high-intensity exercise or breezing; and modifying supplement amounts based at least in part on the implemented exercise regimen for the horse; administering to the horse a dietary supplement containing one or more of Coenzyme Q10 and vitamin E in the modified dietary supplement amounts; ascertaining, with an equine blood-analysis apparatus, or the like, a health state of the horse, the ascertained health state of the horse being indicative of exercise-induced oxidative stress and its sequelae in the horse; and determining, with the computer, a result of a comparison between the ascertained health states of the horse, the compared ascertained health states being indicative of the impact of using Coenzyme Q10 and vitamin E as dietary supplements on exercise-induced oxidative stress and its sequelae in a horse.

### DETAILED DESCRIPTION

It is understood that the embodiments of the present invention are not limited to the particular methodologies, protocols and the like, described herein as they may vary. It is also to be understood the terminology used herein is used for the purpose of describing particular embodiments only and not intended to limit the scope of the present invention. Unless defined otherwise, all technical and scientific terms used herein have the same meaning as commonly understood by one of ordinary skilled in the art to which this invention belongs. While the embodiments disclosed herein pertain to horses, it is appreciated, alternative embodiments of the present invention, may pertain to humans and/or other animals.

The many embodiments of the presently disclosed invention relate in part to using nutritional supplements to achieve greater health and athletic performance within the Equus species of animals in particular-including horses, donkeys, mules, zebras, and/or the like. In some embodiments of the present invention a supplement and/or nutraceutical may be used with and/or beapplied to other animals and/or species. For example, some embodiments of the present invention may be administered to dogs, cats, any type of horse, and/or the like. Some embodiments of the present invention may be administered to, and be effective in treating humans. Supplements and/or nutraceuticals in accordance with embodiments of the present disclosure may be used to as a preventative, rehabilitative, and/or curative measure in treating all equine, including all performance equine during training, after training, when stressed, under high exertion.

Repeated bouts of high intensity exercise also may lead to severe stress resulting in the depletion of vitamin E and endogenous coenzyme Q10 (CoQ10). The fundamental role of CoQ10 in cellular bioenergetics is established in humans and several other species, its role in horses and specifically its relationship with the bioavailability of vitamin E in horses has not been studied. Some embodiments may be used to prevent, cure, treat, and/or rehabilitate, neurological diseases such as equine protozoa! myeloencephalitis (EPM) white muscle disease (WMD), other diseases, and/or the like. By receiving an amount of supplements and/or nutraceuticals in accordance with embodiments of the present disclosure, the horses may receive increased antioxidant effects from increased levels of vitamin E. In one exemplary embodiment, an amount of supplement, nutraceutical, and/or composition in accordance with the present disclosure may be administered to foals to prevent WMD. Compositions, nutraceuticals, supplements, and/or the like of the present invention may comprise Coenzyme Q10, vitamin E, and selenium. Selenium may work in tandem with vitamin E to prevent WMD, or the like. As used herein, the term Coenzyme Q10 and its variants may generally refer to hydrosoluble Coenzyme Q10, or the like. Other types of Coenzyme Q10 may be used, but the dosage amounts required are significantly higher than hydrosoluble Coenzyme Q10, or the like.

Some embodiments of the present disclosure may be used as a preventive and/or curative measure to treat any animal and/or humans with low levels or substantially non-existent levels of vitamin E. For example, supplements, compositions, and/or nutraceuticals in accordance with the present invention may be administered to mares (when pregnant as a preventative measure, or the like), foals, yearlings, through aging equine, or the like. Some of the many embodiments described herein focus on the use of Coenzyme Q10, vitamin E, and their respective variants, for creating increased health and performance among the Equus species. Compositions in accordance with the present disclosure may be referred to the awesome two-some plus, which may comprise Coenzyme Q10, vitamin E, vitamin C, and/or the like.

In some embodiments hydrosoluble Coenzyme Q10 may be included in supplements and/or nutraceuticals in accordance with the present disclosure. In some embodiments, the Coenzyme Q10 may comprise and/or be included in the amount of 100 mg -3.4 g, or the like. Some embodiments focus on increasing the health and athletic performance of horses, using the aforementioned nutrients, with special attention to Thoroughbred race horses, including two-year old Thoroughbreds, some of which are in prime condition for competitive horse-race training. As used throughout the present discussion and in the appended claims, the term “horse” shall be used as definitional shorthand for any member of the Equus species. The particular subject species of animals and the nutrients of special interest as spelled out in the foregoing paragraph, however, are not to be taken as limiting embodiments of the presently disclosed invention. The methods described herein, and as recited in the appended claims, may be applied to any species of organism and using any combination of nutritional supplements. Studies completed by inventors have shown a compound, supplement, nutraceutical, and/or composition in accordance with the present disclosure possesses superior and unexpected properties. For example the studies have demonstrated that Coenzyme Q10 spares vitamin E when administered to equine, or the like. One such study completed by the inventors includes “Tocopherols in Thoroughbred Race Horses: Effect of Coenzyme Q10 Supplementation and Exercise”, published in the Journal of Equine Veterinary Science, Volume 34, Issue 2, Pages 265-269, February 2014, which is incorporated herein by reference in its entirety.

In accordance with exemplary embodiments of the present invention, dietary Coenzyme Q10 (“CoQ10”) and vitamin E may be administered to horses via a horse feed mixture and/or a feeding tube for improving the health and performance of horses, including thoroughbred race horses. Dietary CoQ10 and vitamin E supplementation in accordance with embodiments of the present invention may lead to a marked increase in plasma CoQ10 and vitamin E concentrations, improvements in health and performance, and prevention and/or treatment of many physical and/or neurological disorders. In some embodiments, compositions, supplements, and/or nutraceuticals in accordance with the present invention may be used to prevent and/or treat horses, humans, or other animals for conditions, including physical and/or neurological disorders, caused by deficiencies in vitamin E, which may be naturally occurring or may occur as a result of stress, exercise, and/or the like. In some embodiments, the supplements disclosed herein may also be administered to support the respiratory system of an equine, to support endothelial cell function.

Race horses often participate in high intensity exercise, which may have detrimental effects on the health and performance of the horses. Repeated bouts of high intensity exercise will lead to severe stress resulting in the depletion of endogenous CoQ10, which is the first line of defense among lipophilic antioxidants. The next in line to be depleted would be vitamin E, or tocopherols (α and/or γ-tocopherols), due to continued excessive oxidative stress. Generally, CoQ10 may potentiate the antioxidant effects of vitamin E, and may regenerate vitamin E from its oxidized form. Accordingly, CoQ10 has a sparing effect on vitamin E levels, such that CoQ10 may protect and/or regenerate vitamin E.

A sharp decrease in plasma CoQ10 and vitamin E may occur, however, following bouts of high intensity stress and/or exercise such as breezing sessions, despite the fact that horses continue to receive low dose CoQ10 supplementation. Further, standard horse feed, without supplementation in accordance with the present invention, does not contain enough synthetic or natural vitamin E to support the health of the horse. Other food sources for a horse, such as grass contains very little vitamin E. In horses not receiving any CoQ10-containing dietary supplements, there is a marked decrease in plasma CoQ10 and/or vitamin E concentrations when they are subjected to stress or exercise such as high intensity training, and this could possibly lead to impaired health and poor physical performance.

Exercise-induced pulmonary hemorrhage (EIPH) is a serious problem in thoroughbred race horses or other horses that has been attributed to sustained strenuous exercise. EIPH is associated with poor performance in Thoroughbred race horses that are not medicated with diuretics and not using nasal dilator strips or other drugs. Exercise-induced oxidant stress as shown by increased production of reactive oxygen species (ROS) and free radicals may be present in horses and exercise-induced oxidant stress may be implicated in the pathogenesis of chronic inflammatory, vascular, and airway diseases in horses. Strenuous exercise may cause deleterious effects on pulmonary immune responses. High-intensity exhaustive exercise may lead to a chronic inflammatory state in horses that can affect their physical performance. Inflammation can increase the risk of developing EIPH. The risk of EIPH may be reduced by mitigating or suppressing exercise-induced oxidative stress and its sequelae in race horses by supplementation in accordance with the presentation, for example, supplementation with CoQ10 and vitamin E, and/or other nutraceuticals, or the like.

In accordance with embodiments of the present invention, to protect vitamin E and CoQ10 levels in horses, increase and/or stabilize the bioavailability of vitamin E in horses, and thereby improve their health and physical performance, a dietary supplement composition may be administered to horses. The composition may include CoQ10 and vitamin E, and optionally, other nutrients or vitamins, for general benefit to the horse. The composition may include a high dose of CoQ10 and/or vitamin E and may be administered daily. For example, the composition may comprise from about 250-9000 IUs of vitamin E and from about 5 mg-2500 mg of CoQ10. In one embodiment, the composition may comprise about 1500 IU of vitamin E and about 100 mg of CoQ10. The composition may comprise natural vitamin E and/or synthetic vitamin E. For example, a daily dose of 1200-1600 IU of RRR-alpha tocopheryl acetate (natural vitamin E) may be administered to horses. In exemplary embodiments, administering a daily dose of natural vitamin E may maintain adequate plasma vitamin E in exercising horses. In some embodiments the amounts of the supplements and/or nutraceuticals in accordance with the present invention that are administered for preventative purposes may be different than amounts administered. For example administering a nutraceutical to reestablish vitamin E levels and for treatment dosages may be higher than administering a nutraceutical to prevent deficiencies in vitamin E and the health conditions related to vitamin E deficiency. In some embodiments, horses of different ages and/or weights may be administered different amounts. For example, foals may be administered 250 mg of CoQ10 and an effective amount of vitamin E, which may comprise 1000 IUs, or the like, as a preventative measure, yearlings may be administered a higher amount than foals, and older horses may be administered a higher amount than yearlings. In older horses, 2,000-5000 IUs of vitamin E may be administered as a preventative measure and 4,000-9,000 IUs, in combination with CoQ10, may be administered to restore vitamin E levels and/or cure physical and/or neurological disorders, or the like. In some embodiments, supplements and/or nutraceuticals in accordance with the present disclosure may help promote a healthy immune system, or the like.

In some embodiments, there may be no changes in plasma α- and γ-tocopherol concentrations following high intensity exercise stress due to the sparing action of CoQ10 as the horses receive CoQ10 supplementation. As such, embodiments of the present invention demonstrate the importance of CoQ10 in thoroughbred race horses, and the need for CoQ10 supplementation to mitigate oxidative stress, support physical performance and also help reduce the risk for a number of neurological diseases. Embodiments of the present invention establish that a high dose CoQ10 supplementation is safe and tolerable for thoroughbred race horses.

CoQ10, also known as ubiquinone or ubiquinol, is a vitamin-like compound that has a fundamental role in cellular bioenergetics. CoQ10 is a cofactor in the mitochondrial electron transport chain (respiratory chain), and thus it has an obligatory role in the generation of biological energy in the form of adenosine triphosphate (“ATP”). CoQ10 may function as a mobile redox agent shuttling electrons and protons in the electron transport chain. Furthermore, CoQ10 in its reduced form as the hydroquinone (called “ubiquinol”) is a potent lipophilic antioxidant that is capable of recycling and regenerating other antioxidants such as α-tocopherol (vitamin E) and ascorbate (vitamin C). CoQ10 also has other important functions such as cell signaling and gene expression. CoQ10 is generally present in the blood serum of Thoroughbred race horses. In accordance with exemplary embodiments, providing dietary CoQ10 supplementation to horses results in a marked increase in serum CoQ10 concentrations and may prevent deterioration of the health of a horse after rigorous exercise.

In some embodiments, CoQ10 concentrations are generally affected after stress, such as when a horse participates in a graduated training, low-intensity exercise (“gallop”) and high-intensity exercise (“breeze”). CoQ10 supplementation in accordance with exemplary embodiments of the present invention may result in a marked increase in plasma CoQ10 concentration, but following high intensity exercise there may be a sharp decrease in plasma CoQ10 even if the horses continue to receive CoQ10 supplementation. After receiving a high dose CoQ10 supplement in accordance with the present invention, high intensity exercise may have no or little affect in plasma α- and γ-tocopherol concentrations, and γ-tocopherol may comprise about 5% of α-tocopherol, due to the sparing effect of the CoQ10.

In exemplary embodiments, the decrease in plasma CoQ10 following stress, exercise, for example breezes, may be attributed to severe oxidative stress brought on by more strenuous training. As such, in horses not receiving any CoQ10 supplementation, extreme stress or high intensity exercise may result in a marked depletion of plasma CoQ10 and other tissues too, and this could impair the horse's health and physical performance.

In accordance with exemplary embodiments, CoQ10 and vitamin E may be administered to horses of varying ages. In one embodiment, CoQ10 and Vitamin E may generally be administered to two-year old healthy Thoroughbred race horses. CoQ10 and Vitamin E may be administered to horses by oral dietary supplement containing CoQ10 and Vitamin E. For example, a CoQ10 supplement such as HydroQ-Sorb® (GelTec), or the like, may be administered. CoQ10 may be administered in a dose between 400 mg (“low” CoQ10) and 3.4 g (high CoQ10). The supplements may be administered with feed for the horses and/or by syringe, or the like. In some embodiments, a supplement and/or nutraceutical in accordance with the present disclosure may be administered to horses having naturally occurring low levels of vitamin E, horses that cannot store vitamin E, and/or horses that do not respond to vitamin E supplementation. By administering a supplement and/or nutraceutical in accordance with the present disclosure, for example, comprising CoQ10 and vitamin E, the CoQ10 may spare the depletion of vitamin E and therefore improve the health of these horses or prevent and/or treat various physical and/or neurological disorders caused by vitamin E deficiencies. Many neurological disorders may be linked to genetic or exercise-induced vitamin E deficiencies, and treatment of such disorders by using a nutraceutical and/or supplement in accordance with the present disclosure is contemplated by and within embodiments of the present invention.

In exemplary embodiments, horses that may be administered CoQ10 and Vitamin E for health improvement may include horses in various weights. By way of example, the weight of the horses may range from about 100 kg to about 570 kg with a mean of 545 kg. In some embodiments, the dosage of CoQ10 and Vitamin E may be administered in different doses calculated on a body weight basis. A 100 kg horse may be administered a lower dose than a 570 kg horse. By way of example, CoQ10 may be administered in a ratio of from about 3.486 mg/kg to about 6.239 mg/kg.

Vitamin E is administered in accordance with the present invention, and the health benefits may be observed after the horses are subjected to an exercise regimen. An exercise regimen may include subjecting performance horses to daily 1¼ mile gallop, jumping, rodeo, and/or the like sessions which increased to 1⅜ miles over eight weeks. The exercise regimen may further comprise, over the next nine weeks, after horses are engaged in graduated sessions; allowing the horses to gallop every day and on or about the tenth day, allowing the horses to participate in a ⅛ mile breeze followed by a subsequent ⅛ mile breeze following daily gallop sessions ten days later. The exercise regimen may also comprise a continuation of this progression, for example, two ¼ mile breezes for three weeks and two ⅜ mile breezes for the last three weeks. The exercise regimen may also comprise fully conditioning the horses with 120+days of training,

In exemplary embodiments, when blood plasma is collected and stored frozen at −20° C. until analyses, the analysis of total CoQ10 in plasma (ubiquinone+ubiquinol) may be carried out by high-performance liquid chromatography (“HPLC”) using CoQ6 as the internal standard. Plasma α- and γ-tocopherols may also be determined by HPLC. Data analysis of the blood plasma may be analyzed according to analysis of variance(“ANOVA”), and significance may be set at p<0.05. In accordance with exemplary embodiments, after administration of Co 10 in horses and participation of the horses in an exercise regimen, the baseline plasma CoQ10 concentrations may be comparable at 0.113 and 0.109 μg/mL respectively (See Table 1 below). There may be a variation in baseline plasma γ-tocopherol concentrations that may range from 0 (undetectable) to 0.34 μg/mL with a mean of 0.134 and 0.0808 μg/mL respectively, whereas baseline plasma or α-tocopherol values may be rather closer in proximity at 2.226 and 2.090 μg/mL (See Tables 2 & 3 below).

TABLE 1
Plasma CoQ10 (μg/mL)

| | Baseline | Gallop | Breeze |
|---|---|---|---|
| CoQ10 dose: 1.9 g | 0.113 ± 0.035 | 0.322 ± 0.133 | 0.219 ± 0.062 |
| p | | <0.05 | <0.05 |
| CoQ10 dose: 3.4 g | 0.109 ± 0.019 | 0.813 ± 0.374 | 0.315 ± 0.074 |
| p | | <0.05 | <0.05 |

N = 5 per group

TABLE 2
Plasma γ-Tocopherol (μg/mL)

| | Baseline | Gallop | Breeze |
|---|---|---|---|
| CoQ10 dose: 1.9 g | 0.134 ± 0.130 | 0.124 ± 0.071 | 0.132 ± 0.069 |
| p | | NS* | NS |
| CoQ10 dose: 3.4 g | 0.092 ± 0.080 | 0.064 ± 0.079 | 0.132 ± 0.114 |
| p | | NS | NS |

*Not significant; N = 5 per group

TABLE 3
Plasma α-Tocopherol (μg/mL)

| | Baseline | Gallop | Breeze |
|---|---|---|---|
| CoQ10 dose: 1.9 g | 2.226 ± 0.620 | 2.332 ± 0.696 | 2.134 ± 0.559 |
| p | | NS* | NS |
| CoQ10 dose: 3.4 g | 2.090 ± 1.077 | 1.806 ± 0.718 | 2.074 ± 0.751 |
| p | | NS | NS |

*Not significant; N = 5 per group

In one exemplary embodiment, during the first eight weeks of supplementation with CoQ10 horses may be subjected to a daily performance exercise regimen of gallop, and at the end of this period blood samples may be taken for analyses. After supplementation with CoQ10, plasma CoQ10 values may show a marked increase. For example, plasma CoQ10 values may increase from 0.113 to 0.322 μg/mL or from 0.109 to 0.813 μg/mL. There may be a degree of variability in plasma γ-tocopherol concentrations and little differences between baseline and following the exercise session. Plasma α-tocopherol values were rather tight, and like γ-tocopherol, no differences were observed between the two points.

In some embodiments, the supplementation may be continued indefinitely, or for a specific period of time. For example, supplementation may continue during a time period in which a horse is regularly engaged in exercise activities. In one embodiment, CoQ10 and vitamin E may be administered for a total of 120 days or more during which period the horses may be performing galloping daily and breezing at least six times. Without supplementation of high dose CoQ10 and vitamin E, at the end of this 120 day or more period of supplementation and exercise, there may be a sharp drop in plasma CoQ10 concentrations. For example, plasma CoQ10 concentrations may drop from 0.322 to 0.219 μg/mL or from 0.813 to 0.315 in μg/mL, yet these values may still be above baseline values of 0.113 and 0.109 μg/mL respectively. There may be no significant changes in the case of either α- or γ-tocopherol concentrations in plasma following high intensity exercise session. In exemplary embodiments, although some horses may fail to complete the breeze sessions, no adverse effects of CoQ10 supplementation may be observed in any horse.

In one embodiment, the baseline plasma CoQ10 concentrations in young Thoroughbred race horses may be much lower than that in humans and several other species of animals. The relatively low plasma CoQ10 concentration in horses may be partly attributable to their vegetarian diet. Plasma CoQ10 concentrations may show a marked increase in horses supplemented with CoQ10 (both low dose and high dose) following low intensity slow exercise (gallop) sessions. The CoQ10 dosages in accordance with exemplary embodiments may be similar to therapeutic dosages used in humans, or they may be provided in higher doses. The increase in plasma CoQ10 in horses following CoQ10 and Vitamin E supplementation in low doses may be lower than that following CoQ10 and Vitamin E supplementation in high doses.

In exemplary embodiments, the results yielded by methods of the present invention are completely unexpected. More specifically, following repeated bouts of high-intensity exercise (breeze) following low intensity exercise (gallop), there may a sharp decline in the plasma CoQ10 concentrations in both horses that have received high dose CoQ10 supplementation and horses that have received low dose CoQ10 supplementation, despite the fact that both groups of horses continue to receive daily CoQ10 supplementation. This result is completely unexpected.

Intensive exercise may induce increased generation of reactive oxygen species (“ROS”) and free radicals in horses. Excessive production of free radicals can overwhelm the endogenous antioxidant systems leading to oxidative stress in horses. The endogenous antioxidant defenses are comprised of both enzymatic and non-enzymatic systems. The non-enzymatic antioxidants may include both water-soluble (such as ascorbic acid) and lipid-soluble compounds (such as CoQ10, tocopherols, carotenoids, or the like). CoQ10 in its reduced form as ubiquinol is a potent physiological lipid-soluble antioxidant that functions as the first line of defense under oxidative stress, eventually leading to its depletion. Accordingly, this could explain the marked reduction in plasma CoQ10 concentration following high-intensity exercise due to excessive oxidative stress. This observation may be made in horses receiving a dietary supplement containing CoQ10. In addition, horses that are not getting any dietary supplement containing CoQ10 may also be more severely affected, based upon the mechanism involved in the depletion of CoQ10.

Embodiments of the present invention are highly significant from a practical perspective in that they demonstrate that repeated bouts of high intensity exercise will lead to severe stress resulting in the depletion of endogenous CoQ10 which is the first line of defense among lipophilic antioxidants. High-intensity exercise may result in a marked depletion of endogenous CoQ10 in plasma, which could compromise the health and physical performance of horses. The next in line to be depleted would be α-tocopherol (or both α- and γ-tocopherol) due to continued excessive oxidative stress. In embodiments of the present invention, the oxidative stress may affect the tocopherols after strenuous training. Thus, unless CoQ10 stores are replenished promptly, this may result in a detrimental effect on the health and physical performance of the horses under strenuous training, and this underscores the importance of CoQ10 supplementation to maintain adequate CoQ10 and antioxidant defenses in accordance with the present invention. Embodiments of the present invention demonstrate that high dose CoQ10, and vitamin E supplementation in thoroughbred race horses may be safe for the horses. The deleterious effects of strenuous exercise on pulmonary immune responses may be demonstrated in horses. High intensity exhaustive exercise could lead to a chronic inflammatory state that can also affect a horse's physical performance. The expression of pro-inflammatory cytokine gene expression may be dependent on the intensity of exercise in horses.

Embodiments of the present invention include mitigating or suppressing exercise-induced oxidative stress and its sequelae in race horses with the use of CoQ10 and vitamin E supplementation. In some embodiments “vitamin E” may comprise a complex comprising tocopherols and tocotrienols, or the like. After CoQ10 and vitamin E supplementation, the risk for a number of neurological diseases may be reduced. Baseline values for plasma γ-tocopherol in the present invention may range from 0-0.34 μg/mL, and this may be about 5% of α-tocopherol values. There may be a great deal of variability, and low and high intensity exercise sessions may have no effect on plasma γ-tocopherol concentrations. As opposed to γ-tocopherol, plasma α-tocopherol values may be rather tight after intense exercise, ranging from 1.05 to 3.71 μg/mL, with a baseline mean of 2.158 μg/mL. Like plasma γ-tocopherol, low and high intensity exercise sessions had no effect on plasma or α-tocopherol concentrations either. Endurance exercise has little effect on plasma α-tocopherol in horses following 80 and 160 km race after CoQ10 and vitamin E supplementation.

In accordance with exemplary embodiments of the present invention, oxidative stress must be severe to consume the tocopherols, which may be spared at the expense of CoQ10. Administration of CoQ10 and vitamin E supplements may prevent the depletion of the vitamin E in some horses, even after high intensity physical activity. As such, administration of CoQ10 and vitamin E supplements may reduce the risk and/or occurrence of abnormal neuromuscular function and/or neurological diseases or disorders in horses caused by a deficiency in Vitamin E. In some embodiments, horses may have a genetic disposition such that their bodies can't store vitamin E. In cases where horses, or the like, have a genetic disposition in which their bodies can't store vitamin E, a supplement and/or nutraceutical in accordance with the present invention may be administered continuously and/or the like. In some embodiments, vitamin E deficiencies may produce muscle tremors, neurological head shaking, muscle spasms, and many more.

In some embodiments, CoQ10 may be administered with vitamin E in horses or other animals whose bodies can't store vitamin E, which may be a rare condition. The inclusion of CoQ10 with vitamin E spares the depletion of vitamin E and may be administered to humans, horses, other animals, and/or the like. As such, a composition comprising CoQ10, vitamin E, and other ingredients may be administered to pregnant mares and/or other horses to prevent and/or treat problems caused by vitamin E deficiencies, such as physical and/or neurological disorders, which in some cases may be rare. When administered to pregnant mares, a supplement, composition, nutraceutical, drug and/or the like in accordance with the present disclosure may prevent and/or treat problems caused by vitamin E deficiencies, such as physical and/or neurological disorders in both the pregnant mare and the unborn horse, foal, or the like carried internally by the pregnant mare. In some embodiments a supplement and/or nutraceutical in accordance with the present disclosure may be administered to foals with vitamin E deficiencies and/or the like for preventing and/or treating physical and/or neurological disorders, or the like.

In exemplary embodiments, a supplement and/or nutraceutical in accordance with the present disclosure may be intended for use in major species (horses, dogs, cats, cattle, pigs, turkeys, chickens, or the like) for diseases that occur infrequently and/or in limited geographic areas and in only a small number of animals annually. In some embodiments, dietary and/or nutritional supplement amounts to be administered the horse may be determined, based at least in part on one or more ascertained vital characteristics of the horse (e.g., age, weight, height, gender, breed, size, racing category, performance category, labor category, farming category, and/or the like; with particular embodiments focused on racing Thoroughbreds, performance horses, and/or any horse). In some embodiments, dietary and/or nutritional supplement amounts, nutraceutical amounts, drug amounts, and/or the like may be determined by ascertaining the levels of coenzyme Q10 and vitamin E and prescribing or supplementing an effective amount in accordance with the present disclosure. In particular embodiments, this step may be performed by a computer, such as, without limitation, a general-purpose computer, or the like. In other embodiments, this step may be carried out by use of look-up-tables (LUTs) or the like. As used herein, the term “an effective amount” may comprise determined dietary and/or nutritional supplement amounts to be administered to a horse.

After the dietary supplement amounts are determined via blood analysis, which may be performed by a veterinarian, a credentialed professional, another individual, and/or the like the supplements may be administered to the horse. As always, before administering any supplement, nutraceutical, composition, and/or the like, individuals should seek the advice of a qualified veterinarian or credentialed individual before proceeding with any diagnosis treatment or therapy In particular embodiments, the supplements may comprise Coenzyme Q10, vitamin E, and/or the like. In some embodiments, before and/or after the supplement is administered to the horse, a health status of the horse may be ascertained. In some embodiments, a health state of the horse may be ascertained through any of various methods such as the use of blood chromatography and/or analysis by utilizing serum chromatography systems, methods, and apparatus for the evaluation of equine health states to analyze serum levels of particular nutrients, or the like. It will be appreciated by an ordinary artisan in the field of chromatography that the foregoing references may permit of variations and alternatives, many if not all of which may also be utilized by embodiments of the present invention.

In some embodiments, an ascertained health state may reveal one or more variables associated with an exercise-induced oxidative stress or its sequelae of the horse at or near the time of ascertainment. Non-limiting examples of methods by which a health state of the horse may be ascertained include: analyzing blood plasma concentrations of one or more of Coenzyme Q10, vitamin 10, α-tocopherol, γ-tocopherol, and/or the like; analyzing blood plasma stored frozen at minus 20 degrees Celsius for total concentration of plasma Coenzyme Q10; analyzing the blood plasma with high-performance liquid chromatography (“HPLC”) using Coenzyme Q6 as an internal standard; analyzing blood plasma with high-performance liquid chromatography (“HPLC”) for one or more of α-tocopherols and γ-tocopherols; and applying analysis of variance (“ANOVA”) techniques to results of high-performance liquid chromatography (“HPLC”) applied to blood plasma using a significance test of p<0.05; and/or the like.

In some embodiments, dietary supplement amounts may be modified based at least in part on a health-state determination and the modified nutritional supplement amounts may be administered to the horse. In some embodiments, an additional health state of the horse is determined in accordance with the methods and apparatus identified herein. In some embodiments, a comparison between the health-state and the additional health state may be determined. The determined result of a health-state comparison may comprise, without limitation: comparing baseline plasma concentrations of one or more of Coenzyme Q10, vitamin E, α-tocopherol, γ-tocopherol, and/or the like; comparing baseline plasma concentrations of one or more of Coenzyme Q10, vitamin E, α-tocopherol, γ-tocopherol, and/or the like as ascertained before and after administering one or more of: a 1.9 g dose of Coenzyme Q10, a 3.4 g dose of Coenzyme Q10, and/or the like; and/or the like.

In some embodiments, an exercise regimen may be implemented for the horse by the administrative user of the invention. In some embodiments, an exercise regimen may comprise one or more bouts or sessions of physical exercise, such as graduated training, low-intensity exercise (also called “galloping”), high-intensity exercise (also called “breezing”), and/or the like. In particular non-limiting embodiments, the physical exercise is a one-time session, but in other non-limiting embodiments, the sessions of physical exercise may be repeated over time, and in other embodiments the repeated-over-time sessions may show variations of type and intensity throughout the repetitions. In some embodiments, after the exercise regimen is completed, nutritional supplements may be modified, administered to the horse, and another health-state may be determined in accordance with embodiments of the present disclosure

In some embodiments, an additional comparison may be made between health states of a horse using methods described herein. The compared health states may comprise states before implementation of an exercise regimen and states after implementation thereof. Some embodiments of the invention may loop through the various steps and combinations and sequences thereof. Upon achieving a satisfactory result (e.g., a desired health state of the horse, or a desired result of comparison between health states of the horse), the method may terminate instead of iterating back to an initial step in accordance with the present disclosure. It should be understood that the order, sequence, and timing of any of the steps identified herein are for illustrative purposes only and should not to be read as limiting. Variations of such steps as to order, sequence, and timing, are to be considered as included herein and incorporated hereby into the present disclosure.

In exemplary embodiments, in addition to vitamin E and/or CoQ10, a supplement in accordance with the present invention may comprise Vitamin E, pyrroloquinoline quinone (PQQ), carnitine, tocotrienols, omega 3, magnesium, zinc, folic acid, vitamin C, ribose, curcumin, boswellia, quercetin, selenium, and/or the like. In some embodiments, the composition may be listed to a selected group, excluding all additional elements. Quercetin may be provided in hyrdsoluble form in the amount of 1-5 g, or the like, as a polar auxin transport inhibitor and to increase the efficacy of other elements, such as Coenzyme Q10, vitamin E, vitamin C, and/or the like. In some embodiments a supplement, nutraceutical, and/or the like may comprise boswellia in the amount of 600-800 mg, curcumin in the amount of 600-800 mg, ribose in the amount of 5-10 g, carnitine in the amount of 2-4 g, and quercetin in the amount of 1-5 g, In some embodiments of the present invention, administration of supplements may prevent and care for neurological disorders, physical disorders, and/or the like in performance-equine, other horses, humans, or other animals such as dogs and/or cats. In addition, supplements in accordance with the present disclosure may help maintain healthy muscles and neurological well-being of horses, help protect equine by maintaining healthy responses, and/or the like. In some embodiments, CoQ10 may comprise hydrosoluble (water soluble) CoQ10, or the like. PQQ may be included for mitochondrial support and/or to help reduce damage from free radicals. Carnitine may be included to enhance beta oxidation and shuttle toxins out of a horse. Omega 3 may be included to thin the horse's blood and to reduce inflammation and help with neurological function in the horse. Curcumin, Omega 3, and boswellia may also be included in supplements and/or nutraceuticals in accordance with the present disclosure. Magnesium may be included to stabilize blood pressure. Vitamin C may be included to increase the efficacy of the vitamin E and/or spare the vitamin E from depletion. Selenium may be included to strengthen immune support in a horse. In exemplary embodiments, a composition may comprise vitamin E, vitamin C, selenium, magnesium, CoQ10, and/or the like. In some embodiments, a composition may comprise from 0.5 g to 1.5 g vitamin C, for example, 1 g. In some embodiments, a composition may comprise from 400 mg to 2000 mg CoQ10, for example, 400 mg. In some embodiments, a composition may comprise 0.5 g to 5 g magnesium, for example, 1 gram of magnesium. In some embodiments, a composition may comprise 50 mcg to 50-200 mcg selenium, for example, 200 mcg of selenium. In some embodiments, a composition may comprise from about 5 mg to about 20 g of broad spectrum tocotrienol and/or tocopherol vitamin E complex, for example, 1.5 mg tocotrienols. In some embodiments, a composition may comprise CoQ10, ribose, curcumin, boswellia, vitamin C, vitamin E, magnesium, selenium, and/or the like. In some embodiments, a supplement and/or nutraceutical in accordance with the present invention may be administered to horses of any ages, or the like. In some embodiments, a supplement and/or nutraceutical in accordance with the present invention may be administered to canines of any age for the purposes of prevention and/or treatment of neurological disorders, or the like. In some embodiments, a supplement and/or nutraceutical in accordance with embodiments of the present invention may be administered to felines of any age to prevent and/or cure neurological disorders, or the like. In some embodiments, a supplement and/or nutraceutical in accordance with embodiments of the present invention may be administered to individuals and/or animals of any size and of any age to prevent and/or cure neurological disorders, or the like. In some embodiments, a supplement and/or nutraceutical in accordance with the present invention may be used as a supplement under As a Food Supplement under Association of American Feed Control Officials (AAFCO) for prevention and/or as a Center for Veterinary Medicine (CVM) approved drug and/or nutraceutical for or a treatment of neurological disorders, or the like.

A supplement, nutraceutical, and/or drug in accordance with embodiments of the present invention may help energize and maintain optimum mitochondrial functioning and peak-equine-performance; may help support recovery of the horse after performance events; may help maintain healthy joints and muscles; help maintain a healthy inflammatory response in young race horses during training; and increase the bioavailability of vitamin E (e.g., a complex compressing tocopherols and tocotrienols), and/or the like. In some embodiments, a composition and/or supplement in accordance with embodiments of the present disclosure may be adapted to recycle and/or regenerate oxidized vitamin E, or the like. Many vitamin E supplements consist of natural or synthetic forms of alpha-tocopherol. As used herein, however, vitamin E, may comprise a complex nutrient comprising soluble naturally occurring compounds that form two groups; tocopherols (saturated) and tocotrienols (unsaturated). Each group may comprise four individual isoforms (α, β, γ and δ). As used herein, the term Vitamin E may comprise a complex comprising tocopherols and tocotrienols, and/or the like.

While the foregoing is directed to embodiments of the present invention, other and further embodiments of the invention may be devised without departing from the basic scope thereof. It is also understood that various embodiments described herein may be utilized in combination with any other embodiment described, without departing from the scope contained herein.

What is claimed is:

1. A method for preventing, curing, treating and/or rehabilitating an Equus species with a physical and/or neurological disease, said method comprising administering to the Equus species a composition comprising Coenzyme Q10 and vitamin E.

2. The method of claim 1 wherein the physical and/or neurological disorder is caused by a deficiency in Vitamin E.

3. The method of claim 2 wherein the Vitamin E deficiency is naturally occurring or is a result of stress or exercise.

4. The method of claim 1 wherein the Equus species is suffering from equine protozoal myeloencephalitis, equine motor neuron disease, or white muscle disease.

5. The method of claim 1 wherein the composition is administered to foals to prevent physical and/or neurological disease.

6. The method of claim 1 wherein the Coenzyme Q10 is hydrosoluble Coenzyme Q10.

7. The method of claim 1 further comprising administering one or more additional elements selected from pyrroloquinoline quinone, selenium, boswelia, curcumin, ribose, carnitine, quercetin, vitamin C, Omega 3, zinc, folic acid and magnesium.

8. The method of claim 1 wherein the vitamin E comprises a soluble vitamin E complex of tocotrienols and tocopherols.

9. A method for treating an Equus species with low levels or substantially non-existent levels of Vitamin E, said method comprising administering to the Equus species a composition comprising Coenzyme Q10 and Vitamin E.

10. The method of claim 9 wherein health and performance of the Equus species is increased as compared to health and performance prior to administration of the composition.

11. The method of claim 9 wherein the Equus species is pregnant.

12. The method of claim 9 wherein the Equus species is a foal, yearling or two year old Thoroughbred.

13. The method of claim 9 wherein the Coenzyme Q10 is hydrosoluble Coenzyme Q10.

14. The method of claim 9 wherein the composition is administered via a feed mixture or a feeding tube.

15. The method of claim 9 which inhibits inflammation and/or exercise-induced oxidative stress in the Equus species.

16. The method of claim 9 which decreases risk of developing exercise-induced pulmonary hemorrhage in the Equus species.

17. The method of claim 9 further comprising administering one or more additional elements selected from pyrroloquinoline quinone, selenium, boswelia, curcumin, ribose, carnitine, quercetin, vitamin C, Omega 3, zinc, folic acid and magnesium.

18. The method of claim 9 wherein the vitamin E comprises a soluble vitamin E complex of tocotrienols and tocopherols.

19. A method for treating animals in need of Vitamin E, said method comprising administering to the animal a composition comprising hydrosoluble Coenzyme Q10 and a soluble vitamin E complex of tocotrienols and tocopherols.

20. The method of claim 19 further comprising administering one or more additional elements selected from pyrroloquinoline quinone, selenium, boswelia, curcumin, ribose, carnitine, quercetin, vitamin C, Omega 3, zinc, folic acid and magnesium.
